(19) United States
(12) Patent Application Publication
(10) Pub. No.: US 2026/0334945 A1
(43) Pub. Date: Oct. 29, 2026

(54) ACTUATION DEVICE FOR A MEDICAL INSTRUMENT, AND MEDICAL INSTRUMENT COMPRISING SUCH AN ACTUATION DEVICE

(71) Applicant: Aesculap AG, Tuttlingen (DE)

(72) Inventors: Dominik Seyfried, Koenigsfeld (DE); Hannes Kizenberger, Immendingen (DE)

(21) Appl. No.: 19/707,601

(22) Filed: Jun. 15, 2026

Related U.S. Application Data
(63) Continuation of application No. PCT/EP2024/087301, filed on Dec. 18, 2024.

(30) Foreign Application Priority Data
Dec. 21, 2023 (DE) 10 2023 136 224.0

(51) Int. Cl. A61B 17/068; A61B 17/00; A61B 18/14

(52) U.S. Cl. CPC A61B 17/068; A61B 18/14; A61B 2017/00367

(57) ABSTRACT

An actuation device for a medical instrument includes a main part with a handle element on the main part and an actuation lever. Tool elements on the instrument can be moved via an actuation of the actuation lever from a first position to a second position and/or from the second position to the first position. The actuation lever can be rotated relative to the handle element of the actuation device, and the actuation device is designed such that rotational input movement of the actuation lever is converted into a translational output movement in order to move the tool elements. A rotational axis about which the actuation lever can be rotated is translationally mounted with respect to the main part such that the rotational axis can be translationally displaced by rotational input movement of the actuation lever.

### CROSS-REFERENCE TO RELATED APPLICATIONS

This application is a continuation under 35 U.S.C. § 365 of International Application No. PCT/EP2024/087301, filed on Dec. 18, 2024, and claims priority to German Application No. 10 2023 136 224.0, filed on Dec. 21, 2023. The contents of International Application No. PCT/EP2024/087301 and German Application No. 10 2023 136 224.0 are incorporated by reference herein in their entireties.

### FIELD

The disclosure relates to an actuation device for a medical instrument and to a medical instrument comprising an actuation device. Such a medical instrument is, for example, a surgical instrument, in particular an electrosurgical instrument, for example a vessel sealing instrument, in particular for bipolar vessel sealing.

### BACKGROUND

Devices or systems for bipolar vessel sealing are known in open and laparoscopic surgery, for example in the surgical fields of general surgery, gynecology, urology and thoracic surgery.

In actuation devices known from the prior art, in particular in a high force range required for providing a clamping force, long actuation travels are necessary in order to apply an actuation force. Long actuation travels sometimes result in a wide actuation lever position and reduced operability of such instruments.

### SUMMARY

The present disclosure is based on the object of improving the operability of the actuation device and thus the operability of an instrument comprising such an actuation device.

One embodiment relates to an actuation device for a medical instrument. The actuation device comprises a main part with a handle element provided on the main part and an actuation lever. Tool elements provided at a distal end of the instrument can be moved via an actuation of the actuation lever at a proximal end of the instrument from a first position, in particular a rest position, from distal to proximal to a second position, in particular a working position, and/or from the second position from proximal to distal to the first position. The actuation lever can be rotated relative to the handle element of the actuation device, and the actuation device is designed such that a rotational input movement of the actuation lever is converted, by means of a transmission member that can be translationally displaced along a shaft axis of the main part, into a translational output movement in order to move the tool elements.

A correspondingly designed rocker lever is, for example, provided between the actuation lever and the transmission member, and is pivotably connected both to the actuation lever and to the transmission member, for example via a particular pivot joint. The transmission member is, for example, provided in a guide of the actuation device and is accordingly translationally guided.

According to the present disclosure, a rotational axis about which the actuation lever can be rotated is translationally mounted with respect to the main part such that the rotational axis can be translationally displaced by means of the rotational input movement of the actuation lever. The translational displacement of the rotational axis is preferably a linear translational displacement. The rotational axis is therefore displaced along a straight line.

According to one embodiment, by means of the rotational input movement of the actuation lever, the rotational axis can be translationally displaced parallel to the shaft axis of the main part 18. It is also conceivable that the rotational axis can be displaced vertically or obliquely to the shaft axis of the main part.

According to one embodiment, a bearing member comprising the rotational axis of the actuation lever is mounted in a guide of the main part, in particular in a guide groove of a housing of the main part. The bearing member is, for example, rotationally and translationally mounted in a guide groove formed in a housing of the main part. For example, the guide, in particular the guide groove, runs parallel to the shaft axis. The bearing member, and thus the rotational axis, can therefore be rotationally and translationally displaced in the guide along the shaft axis.

Advantageously, the actuation lever, in particular the bearing member of the actuation lever, comprises a distal guide track and a proximal guide track, wherein the guide tracks slide along at least one pin provided on the main part upon actuation of the actuation lever.

According to one embodiment, the guide tracks are formed by a guide groove, which is in particular curved, and the pin is provided in the guide groove. The width of the guide groove is matched to the diameter of the pin in such a way that the pin extends with play between the distal guide track and the proximal guide track, and the two guide tracks slide along the pin when the actuation lever is actuated.

According to one embodiment, the translational displacement of the rotational axis is carried out according to a shape of the guide groove, which is in particular curved. The translational displacement of the rotational axis is thus determined by the shape of the guide groove.

In a further development of this inventive concept, the pin contacts the guide groove at a particular contact point, and a shape of the guide groove is designed with respect to the rotational axis over the course of the guide groove such that a distance from a particular contact point to the rotational axis increases from the first position of the actuation device to the second position.

Advantageously, by interaction of the pin and the guide groove, a distal counter bearing and a proximal counter bearing are provided, so that the rotational axis is secured in every position against automatic translational displacement without actuation of the actuation lever.

Alternatively, an embodiment could also be conceivable in which the guide groove does not extend along the bearing member of the actuation lever, but rather in the form of two mirror-symmetrical, laterally opposite and mutually facing guide grooves formed in a housing of the actuation device. In this case, the pin is provided on both sides of the bearing member of the actuation lever and slides along the guide grooves in the housing of the actuation device during actuation. A displacement of the rotational axis during the course of actuation could also be implemented in this manner.

According to a further embodiment, the main part comprises a distal and a proximal pin, and wherein the distal pin of the main part is provided on the main part distally relative to the bearing member, and the proximal pin is provided on the main part proximally relative to the bearing member.

In this case, the distal guide track slides along the distal pin, and the proximal guide track slides along the proximal pin. When the actuation lever is actuated from the first position, for example the rest position, to the second position, for example the working position, the distal pin acts as a counter bearing, thereby displacing the rotational axis in the proximal direction. When the actuation lever is actuated from the second position to the first position, the proximal pin acts as a counter bearing, thereby displacing the rotational axis in the distal direction again.

In a further development of this inventive concept, it has been found to be advantageous that the distal pin contacts the distal guide track at a particular contact point, and the proximal pin contacts the proximal guide track at a particular contact point, and the distal guide track is designed such that the distance from the contact point to the rotational axis increases from the first position of the actuation device to the second position, and the proximal guide track is designed such that the distance from the contact point to the rotational axis decreases from the first position of the actuation device to the second position. Accordingly, the distances between the contact points of the distal pin and the distal guide track, and of the proximal pin and the proximal guide track, increase and decrease, respectively, in opposite directions.

Advantageously, the distal and the proximal guide track are designed in coordination with one another such that, by interaction of the distal pin with the distal guide track, a distal counter bearing is provided, and, by interaction of the proximal pin with the proximal guide track, a proximal counter bearing is provided, so that the rotational axis is secured in every position against automatic translational displacement without actuation of the actuation lever. For example, the effective diameter of the bearing member is constant in the region of the guide tracks.

The proximal guide track and distal guide track are coordinated with one another such that movement of the actuation lever is allowed. The bearing member is therefore not wedged between the proximal pin and the distal pin. On the other hand, the proximal guide track and distal guide track are coordinated with one another such that the translational degree of freedom of the rotational axis is blocked by the two pins in every lever position of the actuation lever, and only via an actuation of the actuation lever itself is a guided translational displacement possible.

It can be provided that the actuation device comprises a return element for returning the actuation lever from the second position, in particular the working position, to the first position, in particular the rest position.

Further embodiments relate to a medical instrument, wherein the instrument comprises an actuation device, wherein the actuation device is provided or formed at a proximal end of the instrument so as to interact with tool elements arranged at a distal end of the instrument, and wherein the actuation device is designed to move the tool elements relative to one another.

According to one embodiment, the medical instrument is designed in the form of an electrosurgical instrument and/or in the form of a stapling device and/or comprises an electrosurgical instrument and/or a stapling device.

### BRIEF DESCRIPTION OF THE DRAWINGS

Further advantages are found in the description and the attached drawings. Embodiments of the present disclosure are shown in the drawings and explained in more detail in the following description. In this case, the same reference signs in different drawings denote the same or at least functionally comparable elements. In the description of individual drawings, reference may also be made to elements from other drawings.

FIG. 1 shows an actuation device known from the prior art for a medical instrument;

FIG. 2 shows characteristic actuation profiles of various actuation devices;

FIG. 3a shows a section of an actuation device according to the present disclosure in a first embodiment in a first position;

FIG. 3b shows a section of the actuation device according to the present disclosure in the first embodiment in a transition from the first position to a second position;

FIG. 3c shows a section of the actuation device according to the present disclosure in the first embodiment in the second position; and

FIG. 4 shows an actuation device according to the present disclosure in a further embodiment in a first position.

### DETAILED DESCRIPTION

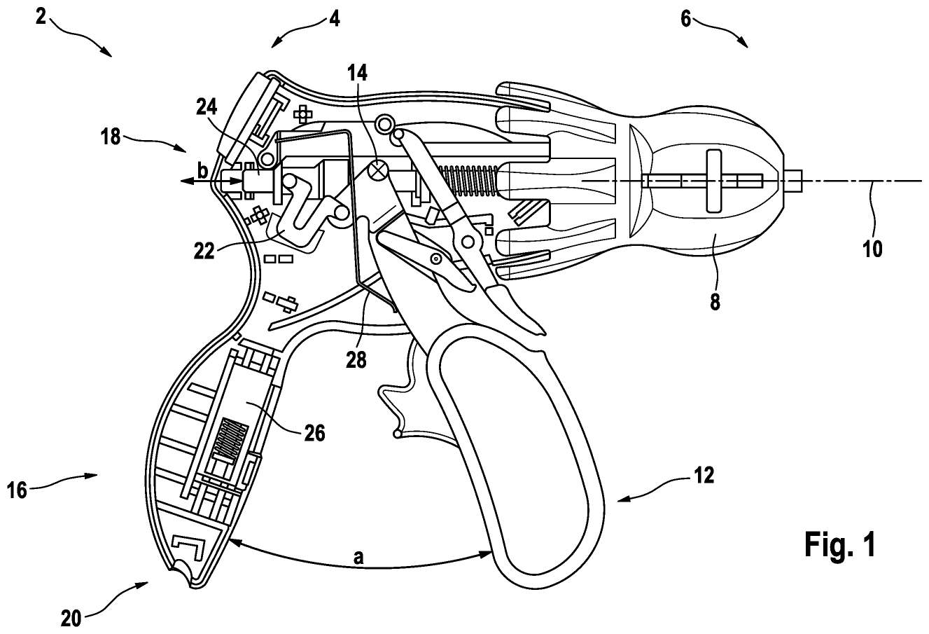

FIG. 1 schematically shows an exemplary embodiment of an actuation device 2 known from the prior art for a medical instrument. The medical instrument is designed, for example, in the form of an electrosurgical instrument (not shown in further detail). This is what is known as a seal-and-cut instrument in which the instrument jaw can be closed and opened by means of the actuation lever 12. In the closed state of the instrument jaw, a seal can be produced on a tissue (usually a hollow organ or blood vessel) by means of electrical coagulation (HF coagulation). In the closed state of the instrument jaw, a cutting blade can also be advanced (“cut”) through the closed instrument jaw by actuation of a further actuation element in order to separate the grasped tissue after coagulation. Alternatively, electrical cutting using HF current and/or ultrasonic cutting is also possible. Such an instrument has a proximal end 4 and a distal end 6.

The actuation device 2 shown in FIG. 1 is, for example, arranged at the proximal end 4 of the instrument. The actuation device 2 is arranged, for example, to be rotatable relative to an elongated shaft 8 of the instrument about a shaft axis 10 defined by said instrument.

An actuation lever 12 projects from the actuation device 2 approximately transversely to the shaft axis 10 and can be pivoted in the proximal direction (and distal direction) about a pivot axis 14 running transversely to the shaft axis 10 in the direction of a fixed handle element 16. The handle element 16 also projects approximately transversely with respect to the shaft axis 10 from a main part 18 of the actuation device 2. The main part 18 forms or comprises, in particular together with the handle element 16 and the shaft 8, a housing of the actuation device 2. Such a housing comprises, for example, two shell-shaped housing parts that, in an assembled state, enclose parts of the actuating mechanism of the actuation device 40. The figures show the housing in an open state, namely with only one shell-shaped housing part.

At a free end 20 of the handle element 16, a connection cable (not shown in further detail) for connection to a power supply device can, for example, be led out.

At the distal end 6 of the instrument, for example, tool elements (not shown in further detail), such as a first tool element and a second tool element, are provided so that they can be moved relative to each other, namely pivoted.

The first tool element is coupled to the actuation lever 12 via a force transmission member (for example, a pull wire) (not shown in further detail), so that as a result of a pivoting movement of the actuation lever 12 in the direction of the handle element 16, the first tool element can be pivoted in the direction of the second tool element.

In FIG. 1, the actuation device 2 is shown in a first position, for example a rest position. In this position, the tool elements provided at the distal end 6 of the instrument, also known as the instrument jaw, are open.

The actuation device 2 can be moved via an actuation of the actuation lever 12 from the rest position from distal to proximal to a second position, for example a working position. In this process, a rotational input movement about the pivot axis 14, cf. double arrow a, of the actuation lever is transmitted to a rocker lever 22. The rocker lever 22 is mounted on the actuation lever 12 on the one hand by means of a pivot joint and, on the other hand, is likewise mounted on a transmission member 24 by means of a pivot joint. The transmission member 24 is provided so that it can be translationally displaced in a corresponding guide groove of the main part 18. The rocker lever therefore performs a movement that comprises a rotational component and a translational component. The rotational input movement a is transmitted via the rocker lever 22 to a translational movement, cf. double arrow b, of the transmission member 24, in which the transmission member 24 receives the translational component of the movement of the rocker lever 22. The transmission member transmits the translational movement b via a force transmission member (for example, a pull wire) (not shown in further detail) to tool elements (likewise not shown in further detail). In the working position, the tool elements provided at the distal end 6 of the instrument, also referred to as the instrument jaw, are closed.

Depending on the force direction and direction of travel of the input movement a, the actuation of the actuation lever 12 leads, for example, to an opening or closing of the tool elements, which are, for example, jaw parts used for vessel sealing.

According to the example shown, upon full actuation of the actuation lever 12 in the proximal direction, the actuation lever 12 reaches a rotation stop 26 in the working position. When the working position is reached, the mechanical clamping force necessary for vessel sealing is built up between the tool elements, in particular the jaw parts. The rotation stop 26 stops the actuation lever 12 in the working position until the actuation lever 12 is manually released from the rotation stop. A return element 28, for example a return spring, acts on the actuation lever 12, so that it is moved back in a distal direction to the rest position shown in FIG. 1, and the tool elements open completely again.

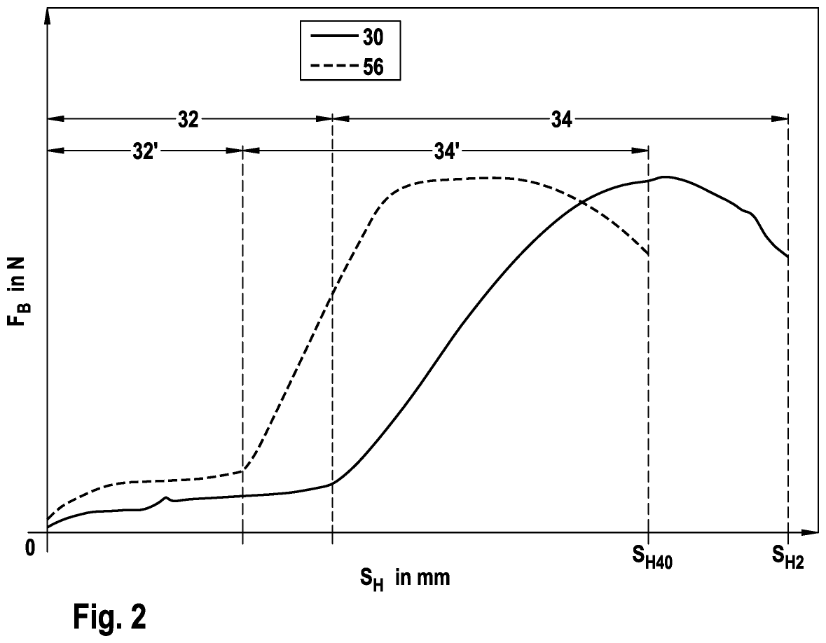

FIG. 2 shows, by way of example, a profile of an actuation force F_{B}, cf. solid line, acting on the actuation lever 12 of the actuation device 2 known from the prior art as a function of an actuation travel S_{H} of the actuation lever 12. In the example, the actuation travel is shown from the first position, which in the example is the rest position, at the axis intersection point (0,0), to a second position, which in the example is the working position. For the actuation device 2, the working position is at S_{H2}.

In the case of an actuation lever 12 known from the prior art, the characteristic actuation force curve 30 shown by a solid line results, in particular when the jaw part is lightly loaded. The actuation force curve 30 can be divided into two portions, portion 32 and portion 34. The first portion 32 comprises, in a relatively low force range, opening or closing of the jaw part. The second portion 34 comprises, in a relatively high force range, the clamping force build-up until the working position is reached.

In the second portion 34, a low increase in the actuation force toward a maximum force can be observed due to the translation profile. After that, a decrease in the actuation force can occur until the working position is reached at S_{H2}. This results in an overall long actuation travel, in particular for the second portion 34 in the high force range. This in turn leads to a wide actuation lever position in the rest position and a reduction in the operability of the actuation device 2.

With reference to FIG. 2 to 5, various embodiments of an actuation device 40 according to the present disclosure are explained below.

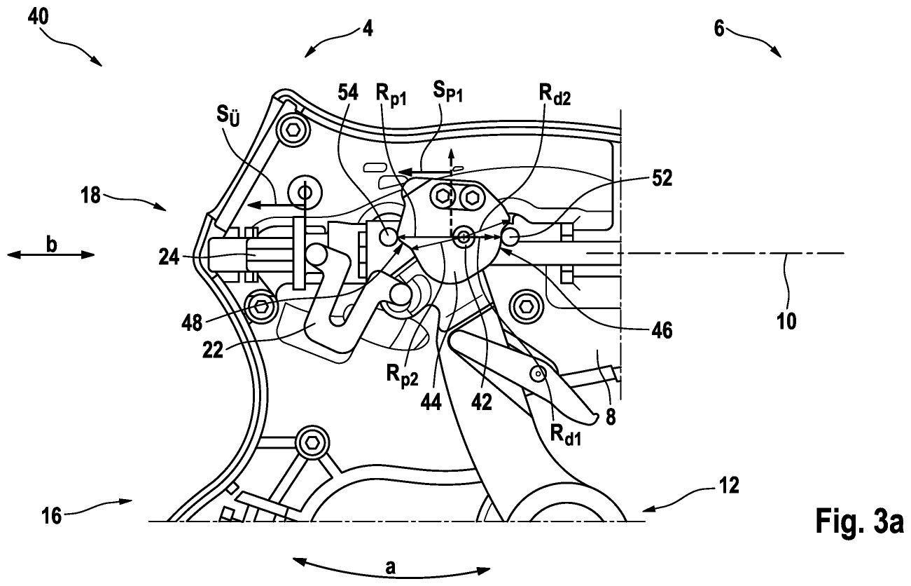

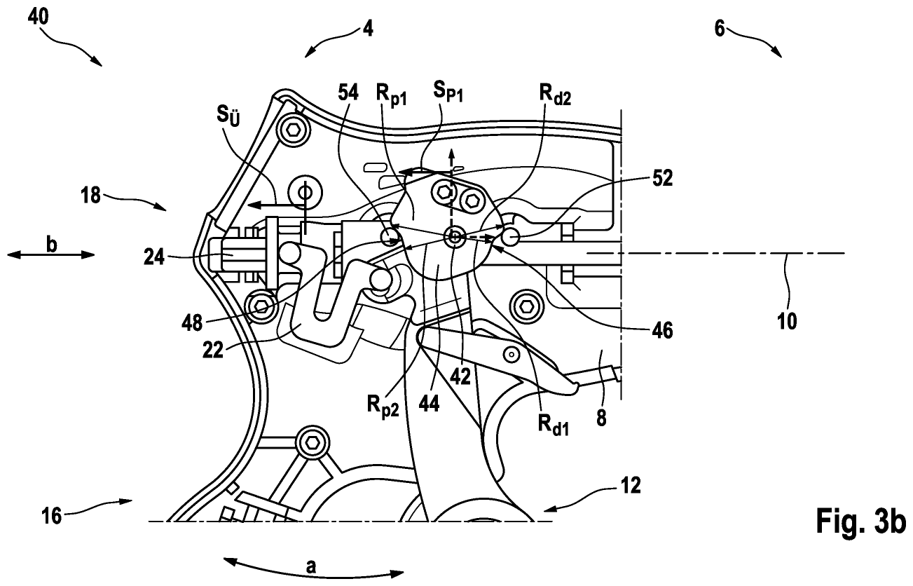

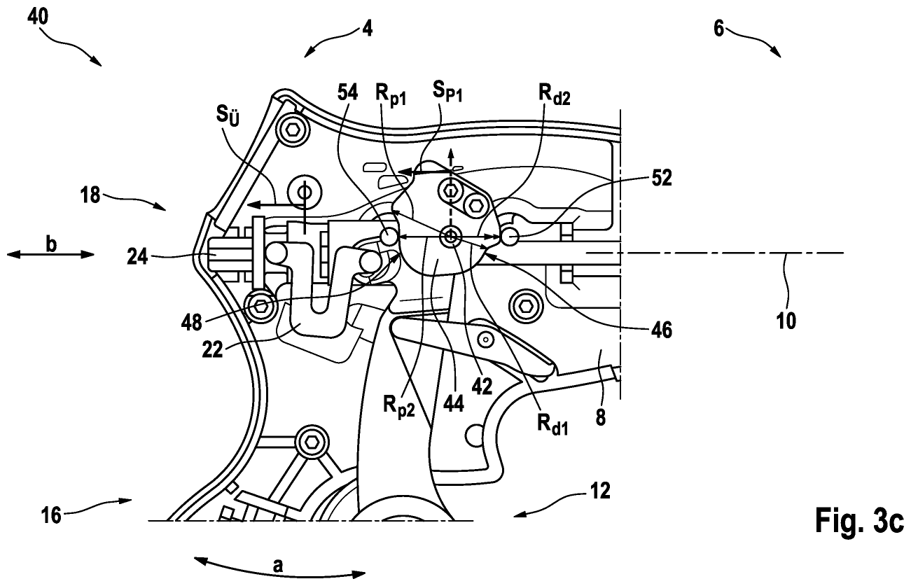

FIG. 3a to 3c show a section of an actuation device 40 according to a first embodiment in a first position, which in the example is a rest position (FIG. 3a), in a transition from the rest position to a second position, which in the example is a working position, from the beginning of a clamping force build-up (FIG. 3b), and in the working position (FIG. 3c).

In the actuation device 40, it is provided that the actuation lever 12 can be rotated about a rotational axis 42. According to the example, the rotational axis 42 is translationally mounted with respect to the main part 18. By means of the rotational input movement a of the actuation lever 12, the rotational axis 42 can be translationally displaced. According to the embodiment shown, the translational displacement of the rotational axis is a linear translational displacement. The rotational axis is therefore displaced along a straight line.

According to the embodiment shown, by means of the rotational input movement a of the actuation lever 12, the rotational axis 42 can be translationally displaced parallel to the shaft axis 10 of the main part 18.

The actuation lever 12 comprises a bearing member 44. The bearing member 44 comprises the rotational axis 42 of the actuation lever 12. The bearing member is, for example, rotationally and translationally mounted in a guide of the main part 18, in particular in a guide groove formed in a housing of the main part 18. According to the example, the guide runs parallel to the shaft axis 10. The bearing member 44, and thus the rotational axis 42, can therefore be rotationally and translationally displaced in the guide along the shaft axis 10. In the example, during actuation from the rest position to the working position, the rotational axis 42 is displaced parallel to the shaft axis 10 over a total travel of approximately 1 mm to 3 mm, in particular 2 mm.

In the example, the actuation lever 12, on the bearing member 44, comprises a distal guide track 46 and a proximal guide track 48. When the actuation lever 12 is actuated, the guide tracks 46, 48 each slide along a pin 50 provided on the main part. In the example, the distal guide track 46 slides along a distal pin 52, and the proximal guide track 48 slides along a proximal pin 54.

The translational displacement of the rotational axis 42 is thus effected via an actuation of the actuation lever, wherein when the actuation lever is actuated from the rest position from distal to proximal to the working position, the distal pin forms a kind of counter bearing and thereby the rotational axis 42 is translationally displaced in the proximal direction, and when the actuation lever is actuated from the working position from proximal to distal to the rest position, the proximal pin forms a kind of counter bearing and thereby the rotational axis 42 is translationally displaced in the distal direction. As explained above, this allows tool elements to be opened and closed, or a clamping force to be built up and released.

In the example, the distal pin 52 contacts the distal guide track 46 at a particular contact point, and the proximal pin 54 contacts the proximal guide track 48 at a particular contact point. The distal guide track 46 is designed such that a distance from the contact point to the rotational axis 42 increases from the first position of the actuation device 40, cf. arrow R_{d1}, to the second position, cf. arrow R_{d2}. The proximal guide track 48 is designed such that a distance from the contact point to the rotational axis 42 decreases from the first position of the actuation device 40, cf. arrow Rₚ₁, to the second position, cf. arrow Rₚ₂.

In the example, the proximal and the distal guide track 46, 48 are designed in coordination with one another such that, by interaction of the distal pin 52 with the distal guide track 46, a distal counter bearing is provided, and, by interaction of the proximal pin 54 with the proximal guide track 48, a proximal counter bearing is provided, so that the rotational axis 42 is secured in every position against automatic translational displacement without actuation of the actuation lever.

The translational displacement of the rotational axis 42 according to the embodiment shown in FIG. 3a to 3c allows for a more rapid increase in force to the maximum force, cf. the characteristic actuation force curve 56 shown as a dashed line in FIG. 2.

The actuation force curve 56 can be divided into two portions, portion 32′ and portion 34′. Starting from the rest position of the actuation device 40, the first portion 32′ comprises, in a relatively low force range, opening or closing of the jaw part during the transition from portion 32′ to portion 34′. The second portion 34′ comprises, in a relatively high force range, the clamping force build-up until the working position of the actuation device 40 is reached.

According to the actuation curves 30, 56 shown, the actuation force F_{B} acting in the range 32, 32′ is higher, in the actuation device 40 according to the present disclosure, than the actuation force F_{B} of the actuation device 2 known from the prior art. In the actuation device 40 according to the present disclosure, the actuation travel S_{H} of the actuation lever 12 in the range 32, 32′ is shorter than in the actuation device 2 known from the prior art. Accordingly, the actuation device 40 according to the present disclosure has a shorter actuation travel S_{H} of the actuation lever 12 required to reach the range of clamping force build-up, cf. range 34′. With approximately the same area of the actuation force curves 30, 56 in the range 32′ and range 32, and thus approximately the same work performed, overall less actuation travel S_{H} of the actuation lever 12 is therefore required.

According to the present disclosure, this is achieved by displacing the position S_{P1} of the rotational axis 42. In the actuation device 40 according to the present disclosure, the position S_{P1} of the rotational axis 42 changes, due to the translational displacement, over the entire actuation travel, and the position S_{Ū} of the transmission member changes as well; see also arrows S_{P1} and S_{Ū} in FIG. 3a to 3c. In comparison, the position of the pivot axis 14 of the actuation device 2 known from the prior art does not change.

The present disclosure thus allows, depending on the displacement or position of the rotational axis 42 and the radii of the guide tracks 46, 48 on the bearing member 44, a reduction of the total actuation travel with approximately constant maximum force. This results in a smaller opening angle of the actuation lever in the rest position, which generally has a positive effect on the handling of the instrument.

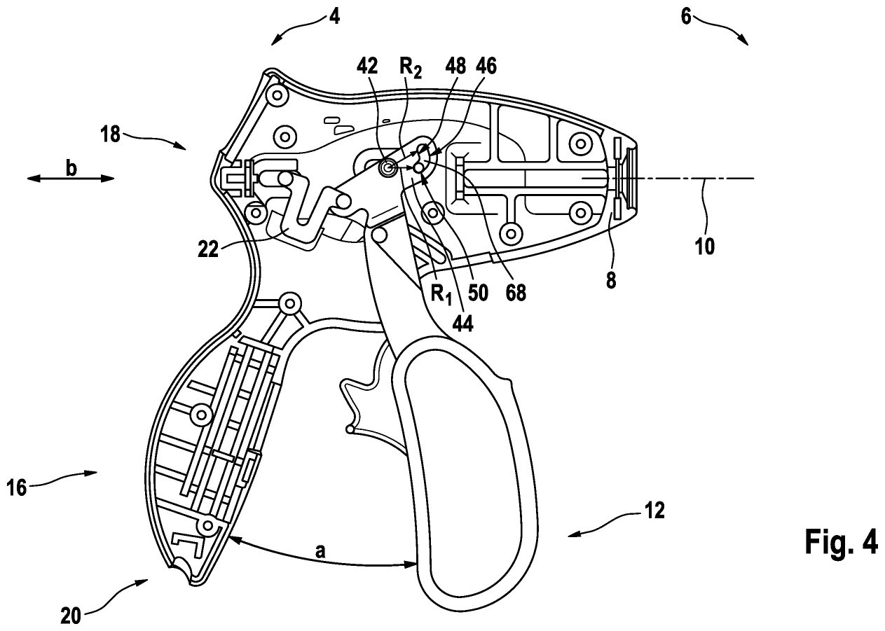

Finally, FIG. 4 shows a further embodiment of the actuation device 40 according to the present disclosure. According to this embodiment, the guide tracks 46, 48 are formed by a curved guide groove 68. The pin 50 is arranged in the guide groove. The distal guide track 46 and the proximal guide track 48 are accordingly provided by the distal and proximal boundary of the guide groove 68.

The width of the guide groove 68 is matched to the diameter of the pin 50 in such a way that the pin 50 is guided with play on both sides between the distal guide track 46 and the proximal guide track 48, and the two guide tracks 46, 48 slide along the pin when the actuation lever is actuated.

The translational displacement of the rotational axis 42 is carried out according to a shape of the curved guide groove 68. The pin 50 contacts the guide groove at a particular contact point. In the example, the shape of the guide groove 68 is designed with respect to the rotational axis 42 over the course of the guide groove 68 such that a distance from a particular contact point to the rotational axis increases from the first position of the actuation device 40, cf. arrow R₁, to the second position, see arrow R₂.

By interaction of the pin 50 and the guide groove 68, a distal counter bearing and a proximal counter bearing are provided, so that the rotational axis 42 is secured in every position against automatic translational displacement without actuation of the actuation lever 12.

In FIG. 4, some elements, for example the transmission member 24 and further elements, are not shown. In the embodiment shown in FIG. 4, these can be provided analogously to the embodiments shown in FIG. 1 and FIGS. 3a-3c. In FIGS. 3a-3c and 4, no return element 28 and no rotation stop 26, cf. FIG. 1, are shown. These elements can likewise be provided in the embodiments shown in FIGS. 3a-3c and FIG. 4, according to the representation in FIG. 1.

### LIST OF REFERENCE SIGNS

2 Actuation device
4 Proximal end
6 Distal end
8 Shaft
10 Shaft axis
12 Actuation lever
14 Pivot axis
16 Handle element
18 Main part
20 Free end (handle element)
22 Rocker lever
24 Transmission member
26 Rotation stop
28 Return element
30 Actuation force curve (prior art)
32 Portion 1
34 Portion 2
40 Actuation device
42 Rotational axis
44 Bearing member
46 Distal guide track
48 Proximal guide track
50 Pin
52 Distal pin
54 Proximal pin
56 Actuation force curve (according to the present disclosure)
58 Profile
60 Profile
62 Range
64 Range
66 Range
68 Guide groove

1. An actuation device for a medical instrument, the actuation device comprising:
a main part with a handle element provided on the main part and an actuation lever; and
a transmission member,
the actuation lever being configured to move tool elements provided at a distal end of the medical instrument via an actuation of the actuation lever at a proximal end of the medical instrument from a first position to a second position, and/or from the second position to the first position,
the actuation lever being rotatable relative to the handle element,
the actuation device being configured such that a rotational input movement of the actuation lever is converted by the transmission member, which is translationally displaceable along a shaft axis of the main part, into a translational output movement in order to move the tool elements, and
a rotational axis about which the actuation lever is rotatable is translationally mounted with respect to the main part such that the rotational axis is translationally displaceable by a rotational input movement of the actuation lever.

2. The actuation device according to claim 1, wherein the rotational axis is translationally displaceable parallel to the shaft axis of the main part in response to the rotational input movement of the actuation lever.

3. The actuation device according to claim 1, further comprising a bearing member that comprises the rotational axis of the actuation lever, the bearing member being mounted in a guide of the main part.

4. The actuation device according to claim 3, wherein the bearing member is mounted in a guide groove of a housing of the main part.

5. The actuation device according to claim 4, wherein the actuation lever comprises a distal guide track and a proximal guide track.

6. The actuation device according to claim 5, wherein the distal guide track and the proximal guide track slide along at least one pin provided on the main part upon actuation of the actuation lever.

7. The actuation device according to claim 6, wherein the guide groove is curved, and the at least one pin is provided in the guide groove.

8. The actuation device according to claim 6, wherein the at least one pin contacts the guide groove at a contact point, and a shape of the guide groove is configured with respect to the rotational axis over the course of the guide groove such that a distance from the contact point to the rotational axis increases from the first position of the actuation device to the second position.

9. The actuation device according to claim 6, wherein, by interaction of the at least one pin and the guide groove, a distal counter bearing and a proximal counter bearing are provided, so that the rotational axis is secured in every position against automatic translational displacement without actuation of the actuation lever.

10. The actuation device according to claim 5, wherein the bearing member comprises the distal guide track and the proximal guide track.

11. The actuation device according to claim 5, wherein the distal guide track and the proximal guide track are formed by the guide groove.

12. The actuation device according to claim 5, wherein the main part comprises a distal pin and a proximal pin.

13. The actuation device according to claim 12, wherein the distal pin is provided on the main part distally relative to the bearing member, and the proximal pin is provided on the main part proximally relative to the bearing member.

14. The actuation device according to claim 12, wherein:
the distal pin contacts the distal guide track at a first contact point,
the proximal pin contacts the proximal guide track at a second contact point,
the distal guide track is configured such that a distance from the first contact point to the rotational axis increases from the first position of the actuation device to the second position, and
the proximal guide track is configured such that a distance from the second contact point to the rotational axis decreases from the first position of the actuation device to the second position.

15. The actuation device according to claim 12, wherein the distal guide track and the proximal guide track are configured in coordination with one another such that, by interaction of the distal pin with the distal guide track, a distal counter bearing is provided, and, by interaction of the proximal pin with the proximal guide track, a proximal counter bearing is provided, so that the rotational axis is secured in every position against automatic translational displacement without actuation of the actuation lever.

16. The actuation device according to claim 4, wherein translational displacement of the rotational axis is carried out according to a shape of the guide groove

17. The actuation device according to claim 1, further comprising a return element for returning the actuation lever from the second position to the first position.

18. A medical instrument comprising the actuation device according to claim 1, wherein the actuation device is provided or formed at a proximal end of the medical instrument so as to interact with tool elements arranged at a distal end of the medical instrument, and wherein the actuation device is configured to move the tool elements relative to one another.

19. The medical instrument according to claim 18, wherein the medical instrument is or comprises an electrosurgical instrument and/or a stapling device.
